(19) United States
(12) Patent Application Publication
(10) Pub. No.: US 2015/0224287 A1
(43) Pub. Date: Aug. 13, 2015

(54) Central Venous Catheter Kit

(71) Applicant: Corvivo, Inc., Coquitlam (CA)

(72) Inventors: Xiaoming Bian, Dalian (CN); Frank Zheng, Kirkland, WA (US)

(21) Appl. No.: 14/621,351

(22) Filed: Feb. 12, 2015

Related U.S. Application Data
(60) Provisional application No. 61/939,177, filed on Feb. 12, 2014.

(51) Int. Cl. A61M 25/09; A61M 25/06

(52) U.S. Cl. CPC A61M 25/09041; A61M 25/065; A61M 2025/0687

(57) ABSTRACT

A central venous catheter kit comprises a medical instrument and a catheter for use during catheterization. The medical instrument includes a syringe having a barrel and a plunger. A needle extends axially from the syringe. The needle has a lumen which is in fluid communication with the barrel of the syringe. There is a dilator disposed about the needle and a guidewire substantially disposed within the syringe. There is a guide for advancing the guidewire out of the syringe through the lumen of the needle. The catheter has a lumen and there is a guidewire disposed within the lumen of the catheter. The guidewire substantially disposed within the syringe and the guidewire disposed within the lumen of the catheter are each provided with a connector to allow the guidewires to be coupled.

This application claims priority to and claims benefit of and priority to U.S. Provisional Application Ser. No. 61/939,177 filed Feb. 12, 2014 entitled Central Venous Catheter Kit, which is hereby incorporated herein by reference in its entirety.

### BACKGROUND OF THE INVENTION

1. Field of the Invention

The present invention relates to a central venous catheter kit and, in particular, to a central venous catheter kit comprising integrated components.

2. Description of the Related Art

Conventional central venous catheter kits generally comprise at least four separate components, namely, a syringe coupled to a needle having a longitudinal lumen, a guidewire, a dilator, and a central venous catheter. The needle is inserted into a patient and blood is aspirated into the syringe through the lumen of the needle to confirm that the needle has punctured a blood vessel, e.g. the subclavian vein. The syringe is then decoupled from the needle and the guidewire is inserted into the blood vessel through the lumen of the needle. The needle is withdrawn following the insertion of the guidewire into the blood vessel. This allows the dilator to be placed over the guidewire and introduced into the puncture site. The dilator is used to dilate tissue around the guidewire to facilitate catheterization and is withdrawn prior to the catheter being placed over the guidewire and inserted into the blood vessel. The guidewire is then withdrawn from the patient, leaving the central venous catheter in the blood vessel. This method of catheterization is known as the Seldinger technique.

It is also known to provide integrated instruments for use during catheterization. For example, U.S. Pat. No. 5,257,979 which issued on Nov. 2, 1993 to Jagpal, discloses a medical instrument for use with a syringe during catheterization. The medical instrument comprises a needle, a hub and a dilator. The needle has an elongate shaft including a hub end and a beveled end for puncturing a blood vessel. There is a first longitudinal lumen extending along a length of the needle shaft, and there is a second longitudinal lumen extending along a length of the needle shaft parallel to the first longitudinal lumen. A hub adapted to engage the syringe is attached to the needle at the hub end thereof. The hub has a first bore in fluid communication with the first longitudinal lumen of the needle to permit fluid communication with the syringe. The hub also has a second bore in fluid communication with the second longitudinal lumen of the needle to allow for selective insertion of a guidewire into the blood vessel through the second longitudinal lumen. There is a dilator circumferentially disposed about the needle. The dilator extends from the hub to adjacent the bevelled end of the needle.

The bevelled end of the needle is used to puncture a blood vessel, e.g. the subclavian vein. Puncturing of the blood vessel is confirmed by aspirating blood into the syringe through the first longitudinal lumen of the needle and the first bore in the hub. A guidewire is then inserted into the blood vessel through the second bore in the hub and the second longitudinal lumen of the needle. The dilator is then moved along the guidewire into the puncture site as the needle and syringe are simultaneously removed from the puncture site. The dilator is rotated in the puncture site and then removed from the puncture site. This opens up the puncture site to facilitate the introduction of the catheter along the guidewire and into the blood vessel during catheterization.

There however remains a need for an improved central venous catheter kit which allows for simplified and safe catheterization.

### SUMMARY OF THE INVENTION

There is provided a central venous catheter kit comprising a medical instrument and a catheter for use during catheterization. The medical instrument includes a syringe having a barrel and a plunger. A needle extends axially from the syringe. The needle has a lumen which is in fluid communication with the barrel of the syringe. There is a dilator disposed about the needle and a guidewire substantially disposed within the syringe. There is a guide for advancing the guidewire out of the syringe through the lumen of the needle. The catheter has a lumen and there is a guidewire disposed within the lumen of the catheter. The guidewire substantially disposed within the syringe and the guidewire disposed within the lumen of the catheter are each provided with a connector to allow said guidewires to be coupled. The connectors may be formed of a magnetic material.

The medical instrument may include a channel in the barrel of the syringe. The guidewire substantially disposed within the syringe may extend along the channel. The medical instrument may include a detent to limit how far the plunger can be withdrawn from the barrel so the plunger does not interfere with the guidewire. The dilator of the medical instrument may include longitudinal lines of weakness and longitudinal gaps which are aligned with the longitudinal lines of weakness. The guidewire disposed within the lumen of the catheter may extend axially beyond the lumen.

In a first embodiment of the central venous catheter kit, the medical instrument may include a first channel and a second channel in the barrel of the syringe. The guidewire substantially disposed within the syringe may extend along the first channel and the second channel. There may be a through hole in the plunger of the syringe. The guidewire substantially disposed within the syringe may extend along the first channel, through the through hole and along the second channel.

In a second embodiment of the central venous catheter kit, the barrel of the syringe of the medical instrument may include an inner barrel and an outer barrel. The guidewire substantially disposed within the syringe may extend between the inner barrel and the outer barrel. There may be a through hole in the plunger of the syringe. The guidewire substantially disposed within the syringe may extend in a U-shaped configuration within the syringe and through the through hole.

In a third embodiment of the central venous catheter kit, the barrel of the syringe of the medical instrument may include an inner barrel and an outer barrel. The guidewire substantially disposed within the syringe may extend between the inner barrel and the outer barrel. The guidewire substantially disposed within the syringe may extend in a U-shaped configuration within the syringe and around the plunger.

In a fourth embodiment of the central venous catheter kit, the needle of the instrument may include a first lumen and a second lumen which is substantially parallel to the first lumen. The guidewire substantially disposed within the syringe may be advanced out of the syringe through the first lumen of the needle.

The catheter of the central venous catheter kit may include a lumen and a guidewire disposed within the lumen. The guidewire disposed within the lumen of the catheter may extend axially beyond the lumen.

### BRIEF DESCRIPTIONS OF DRAWINGS

The invention will be more readily understood from the following description of the embodiments thereof given, by way of example only, with reference to the accompanying drawings, in which:

FIG. 1 is a perspective view of a first embodiment of a medical instrument for use during catheterization;

FIG. 2 is another perspective view of the first embodiment of the medical instrument rotated approximately forty-five degrees from the position shown in FIG. 1;

FIG. 3 is a cross-sectional view taken along lines A-A of FIG. 1 showing a plunger of the first embodiment of the medical instrument in a retracted position;

FIG. 4 is another cross-sectional view, similar to FIG. 3, showing the plunger of the first embodiment of the medical instrument in an extended position;

FIG. 4A is an enlarged view of a portion of FIG. 4 showing a seal of the first embodiment of the medical instrument;

FIG. 5 is a cross-sectional view taken along lines B-B of FIG. 1;

FIG. 6 is a perspective view of a second embodiment of a medical instrument for use during catheterization;

FIG. 7 is another perspective view of the second embodiment of the medical instrument rotated approximately forty-five degrees from the position shown in FIG. 6;

FIG. 8 is a cross-sectional view taken along lines C-C of FIG. 6 showing a plunger of the second embodiment of the medical instrument in a retracted position;

FIG. 9 is another cross-sectional view, similar to FIG. 8, showing the plunger of the second embodiment of the medical instrument in an extended position;

FIG. 10 is a cross-sectional view taken along lines D-D of FIG. 6;

FIG. 11 is a perspective view of a third embodiment of a medical instrument for use during catheterization;

FIG. 12 is another perspective view of the third embodiment of the medical instrument rotated approximately forty-five degrees from the position shown in FIG. 11;

FIG. 13 is a cross-sectional view taken along lines E-E of FIG. 11 showing a plunger of the third embodiment of the medical instrument in a retracted position;

FIG. 14 is another cross-sectional view, similar to FIG. 13, showing the plunger of the third embodiment of the medical instrument in an extended position;

FIG. 15 is a cross-sectional view taken along lines F-F of FIG. 11;

FIG. 16 is a side elevation view of a dilator of the first embodiment of the medical instrument for use during catheterization;

FIG. 17 is a cross-sectional view taken along lines G-G of FIG. 16;

FIG. 18 is a cross-sectional view taken along lines H-H of FIG. 16;

FIG. 19 is a side elevation view of a first embodiment of an improved catheter for use with the first embodiment of the medical instrument during catheterization;

FIG. 20 is a sectional view of the catheter of FIG. 19;

FIG. 21 is a side elevation view showing a guidewire of the first embodiment of the medical instrument and a guidewire of the catheter of FIG. 19 illustrating said guidewires decoupled;

FIG. 22 is a side elevation view showing the guidewire of the first embodiment of the medical instrument and the guidewire of the catheter of FIG. 19 illustrating said guidewires coupled;

FIG. 23 is a partially sectional view showing the first embodiment of the medical instrument being used to puncture a blood vessel;

FIG. 24 is a partially sectional view showing the guidewire of the first embodiment of the medical instrument being inserted into the blood vessel;

FIG. 25 is a partially sectional view showing the dilator of the first embodiment of the medical instrument being inserted into tissue surrounding the guidewire;

FIG. 26 is a partially sectional view showing the dilator of the first embodiment of the medical instrument being rotated to dilate the tissue surrounding the guidewire;

FIG. 27 is a partially sectional view showing the catheter of FIG. 19 being coupled to the guidewire of the first embodiment of the medical instrument;

FIG. 28 is a partially sectional view showing the catheter of FIG. 19 being moved along the guidewire of the first embodiment of the medical instrument;

FIG. 29 is a partially sectional view showing the dilator of the first embodiment of the medical instrument being removed from tissue surrounding the guidewire;

FIG. 30 is a partially sectional view showing the dilator of the first embodiment of the medical instrument being removed from tissue surrounding the guidewire;

FIG. 31 is a partially sectional view showing the catheter of FIG. 19 being moved along the guidewire of the first embodiment of the medical instrument and into the blood vessel;

FIG. 32 is a partially sectional view showing the catheter of FIG. 19 inserted into the blood vessel with the guidewire thereof removed;

FIG. 33 is a sectional view showing a fourth embodiment of a medical instrument being used to puncture a blood vessel;

FIG. 34 is a sectional view showing a guidewire of the fourth embodiment of the medical instrument being inserted into the blood vessel;

FIG. 35 is a partially sectional, side elevation view showing another embodiment of a guidewire of the first embodiment of the medical instrument and a guidewire of a second embodiment of an improved catheter illustrating said guidewires decoupled;

FIG. 36 is a partially sectional, side elevation view showing the guidewire of the first embodiment of the medical instrument of FIG. 35 and the guidewire of the catheter of FIG. 35 illustrating said guidewires coupled;

FIG. 37 is a partially sectional, side elevation view showing yet another embodiment of a guidewire of the first embodiment of the medical instrument and a guidewire of a third embodiment of an improved catheter illustrating said guidewires decoupled; and

FIG. 38 is a partially sectional, side elevation view showing the guidewire of the first embodiment of the medical instrument of FIG. 37 and the guidewire of the catheter of FIG. 37 illustrating said guidewires coupled.

### DESCRIPTIONS OF THE PREFERRED EMBODIMENTS

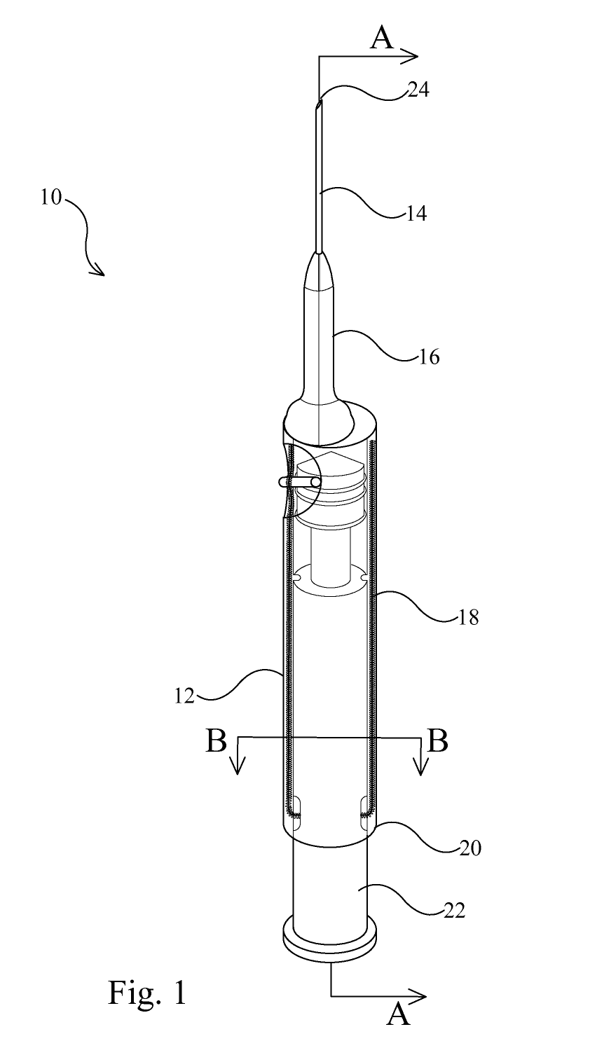

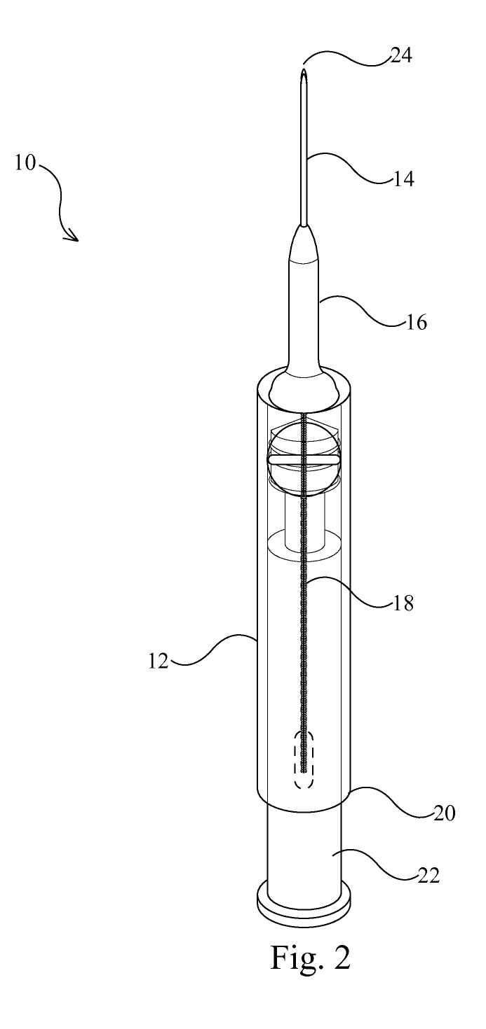

Referring to the drawings and first to FIGS. 1 and 2, there is shown a first embodiment of an improved medical instrument 10 for use during catheterization. The medical instrument 10 generally comprises a syringe 12, a needle 14, a dilator 16, and a guidewire 18. The syringe 12 includes a barrel 20 and a plunger 22 which is reciprocatingly received by the barrel 20 as is conventional. The barrel 20 of the syringe 12 is substantially cylindrical and translucent in this example. The needle 14 extends axially outward from the syringe 12 and is in fluid communication with an interior of the barrel 20 of the syringe 12 as is conventional. The dilator 16 fits over at least a part of the needle 14 with a tip 24 of the needle 14 being remote of the dilator 16. The tip 24 of the needle is bevelled in this example. The guidewire 18 is substantially disposed within the syringe 12.

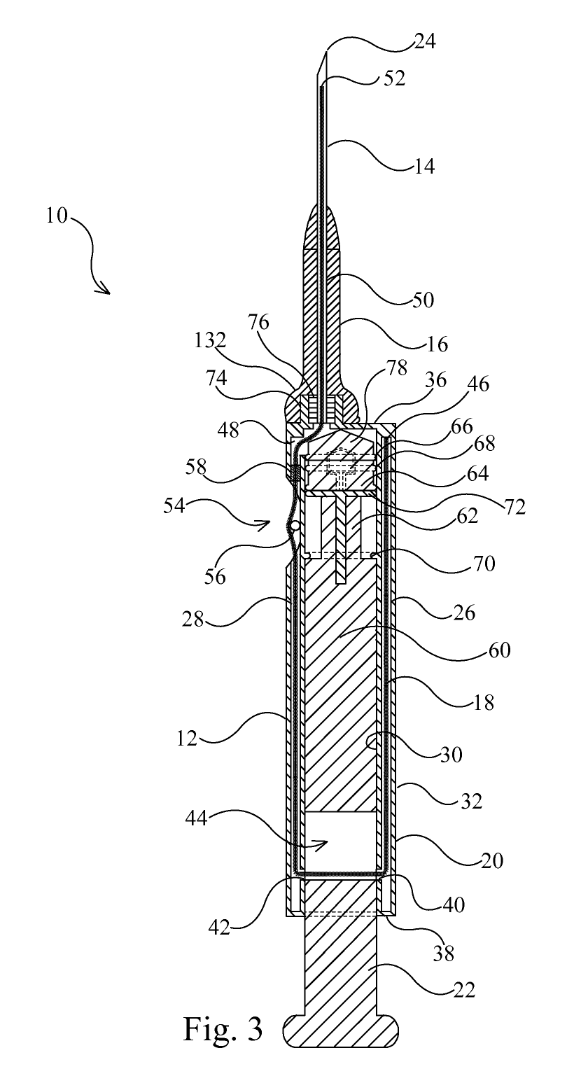

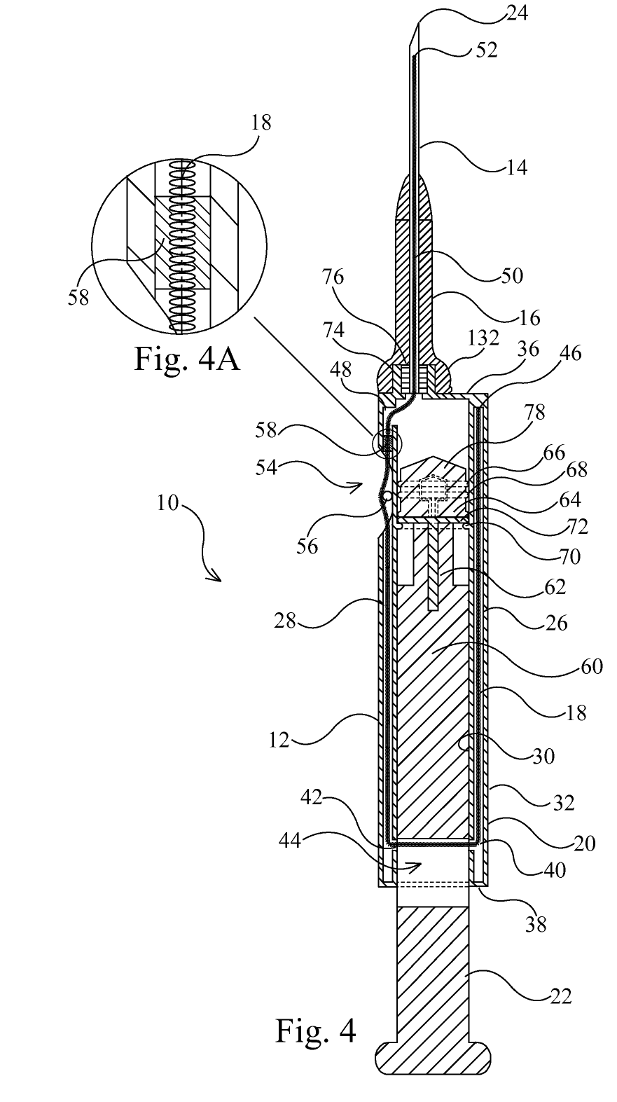

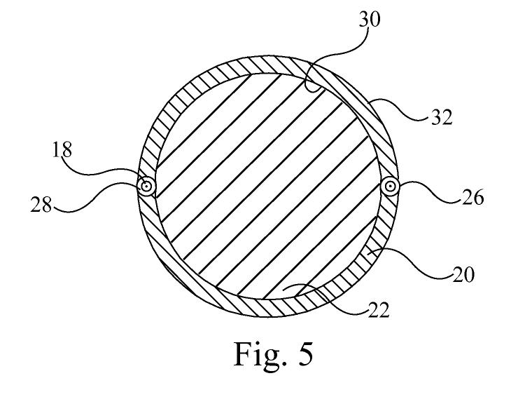

Referring now to FIGS. 3 and 4, in the first embodiment of the medical instrument 10, there is a first channel 26 and a second channel 28 which are each disposed between an inner wall 30 and an outer wall 32 of the barrel 20 of the syringe 12. The first channel 26 and the second channel 28 extend longitudinally along the barrel 20 of the syringe 12 and, as best shown in FIG. 5, the first channel 26 and the second channel 28 are opposite to one another. Referring back to FIGS. 3 and 4, the first channel 26 and the second channel 28 extend from near a nozzle end 36 of the syringe 12 to near a plunger end 38 of the syringe 12. There is a first transverse bore 40 in the inner wall 30 of the barrel 20 of the syringe 12. The first transverse bore 40 in the inner wall 30 of the barrel 20 is near the plunger end 38 of the syringe 12 and allows for communication between the first channel 26 and the interior of the barrel 20. There is a second transverse bore 42 in the inner wall 30 of the barrel 20 of the syringe 12. The second transverse bore 42 in the inner wall 30 of the barrel 20 is near the plunger end 38 of the syringe 12 and allows for communication between the second channel 28 and the interior of the barrel 20. There is a transverse through hole 44 in the plunger 22 of the syringe 12.

A first end 46 of the guidewire 18 is disposed within the first channel 26 near the nozzle end 36 of the syringe 12. The guidewire 18 extends along the first channel 26 and through the first transverse bore 40 in the inner wall 30 of the barrel 20, through the through hole 44 in the plunger 22, and through the second transverse bore 42 in the inner wall 30 of the barrel 20 and along the second channel 28. The guidewire 18 also extends along the second channel 28 from the second transverse bore 42 in the inner wall 30 of the barrel 20 to a third transverse bore 48 in the inner wall 30 of the barrel 20. The third transverse bore 48 in the inner wall 30 of the barrel 20 is near the nozzle end 36 of the syringe 12 and allows for communication between the second channel 28 and the interior of the barrel 20. The guidewire 18 extends through the third transverse bore 48 in the inner wall 30 of the barrel 20 and into the interior of the barrel 20 then into a lumen 50 of the needle 14. In this example, a second end 52 of the guidewire 18 is shown approximately one centimeter away from the tip 24 of the needle 14.

There is an opening 54 in the outer wall 32 of the barrel 20 of the syringe 12. The opening 54 in the outer wall 32 of the barrel 20 exposes the guidewire 18 as it extends along the second channel 28 in the barrel 20. A guide in the form of roller 56 extends across the opening 54 in the outer wall 32 of the barrel 20 and the guidewire 18 extends over the roller 56. The roller 56 facilitates manipulation of the guidewire 18. There is a seal 58 disposed in the second channel 28 in the barrel 20 between the opening 54 in the outer wall 32 of the barrel 20 and the third transverse bore 48 in the inner wall 30 of the barrel 20. The seal 58 is shown best in FIG. 4A and, in this example, is a rubber seal which functions to prevent blood or other bodily fluids from leaking out of the interior of the barrel 20.

The plunger 22 of the syringe 12 includes a body 60, a neck 62, and a head 64. The body 60 and the head 64 of the plunger 22 are each greater in girth as compared to the neck 62 of the plunger 22. The body 60 and the neck 62 of the plunger 22 are unitary, in this example, and are formed of plastic. The head 64 of the plunger 22 is formed of rubber and is friction fitted to the neck 62 of the plunger 22 in this example. There are circumferential ribs 66 and 68 which extend about the head 64 of the plunger 22. The circumferential ribs 66 and 68 are substantially annular, in this example, and provide resistance when the plunger is being moved between the retracted position, which is shown in FIG. 3, and the extended position which is shown in FIG. 4. There is a circumferential rib 70 on the inner wall 30 of the barrel 20 which is substantially annular, in this example, and extends about the inner wall 30 of the barrel 20. The circumferential rib 70 may require a 0.2-0.3 millimeter mold undercut. The circumferential rib 70 functions as a detent to generally prevent the plunger 22 from being withdrawn completely from the barrel 20 and, more specifically, to limit how far the plunger 22 can be withdrawn from the barrel 20 to ensure that the plunger 22 does not interfere with the guidewire 18. A plate 72, upon which the head 64 of the plunger 22 sits, abuts against the circumferential rib 70 on the inner wall 30 of the barrel 20 to limit how far the plunger 22 can be withdrawn from the barrel 20 as best shown in FIG. 4. The through hole 44 in the plunger 22 is large enough so that the plunger 22 may be moved between the retracted position and the extended position without interfering with the guidewire 18.

The syringe 12 further includes a nozzle 74 at the nozzle end 36 thereof. A hub 76 of the needle 14 is received by the nozzle 74 and the dilator 16 is releasably fitted over the nozzle 74. The guidewire 18 is accordingly able to extend from the interior of the barrel 20 of the syringe 12 through the nozzle 74 of the syringe 12 and into the lumen 50 of the needle 14. A tip 78 of the head 64 of the plunger 22 is conical so that the head 64 of the plunger 22 does not interfere with the guidewire 18 when the plunger 22 of the syringe 12 is in the retracted position as best shown in FIG. 3.

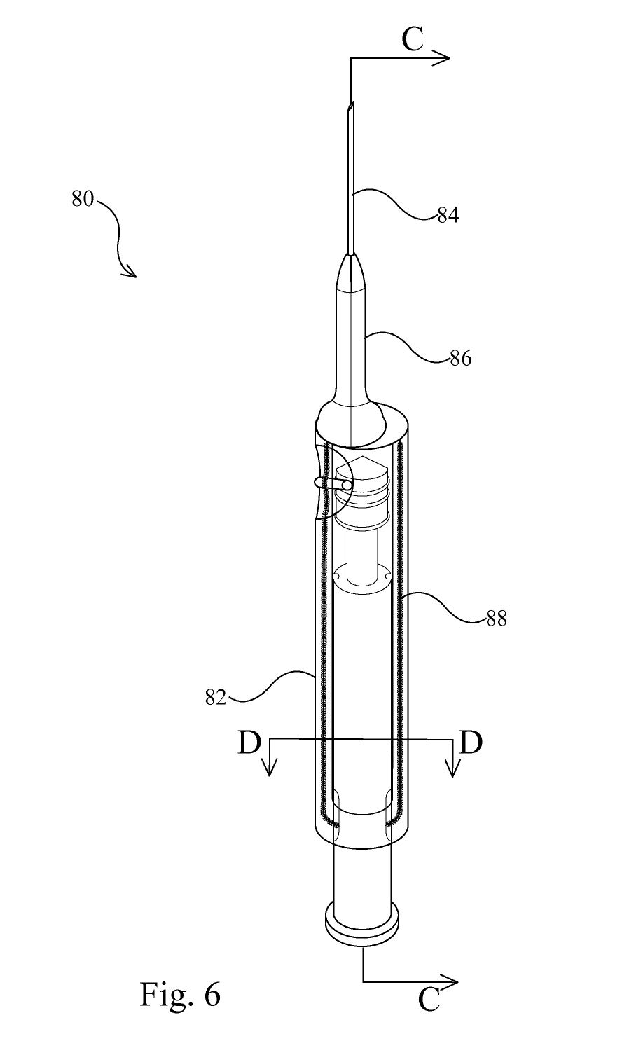

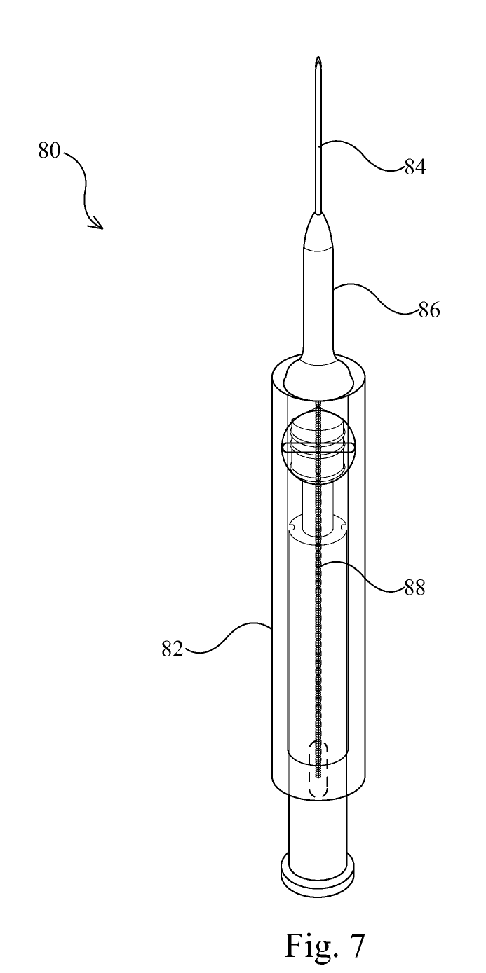

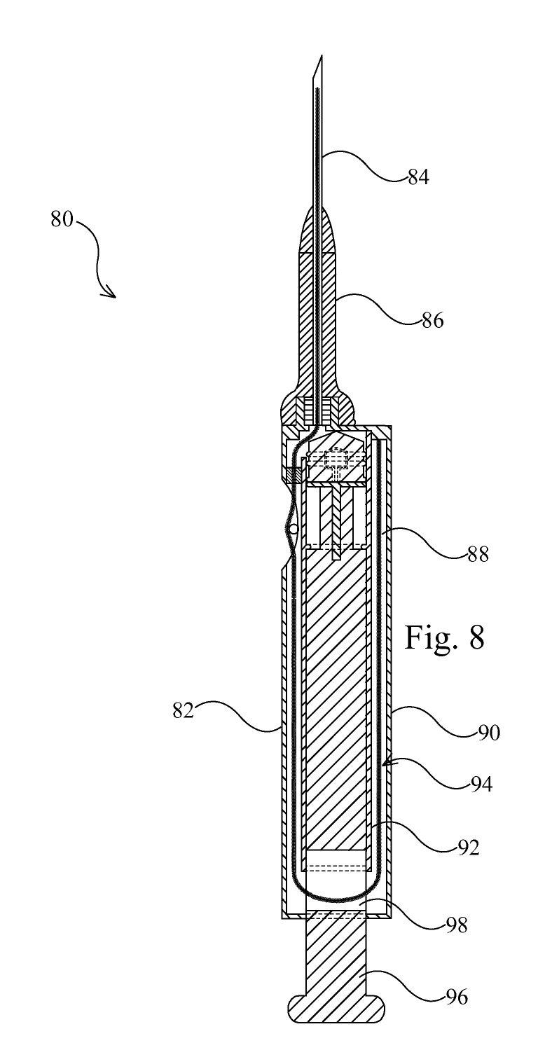

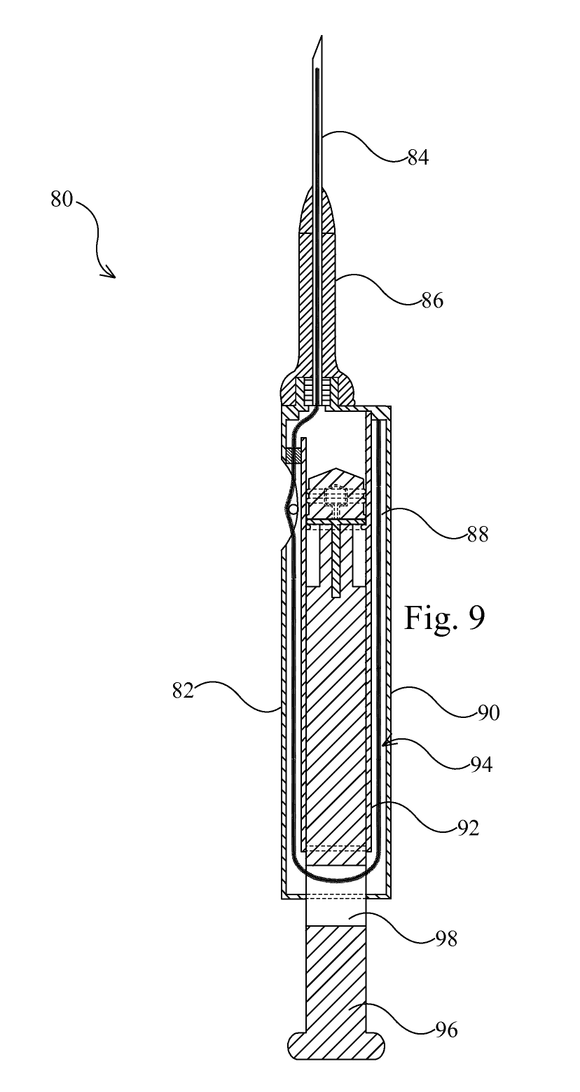

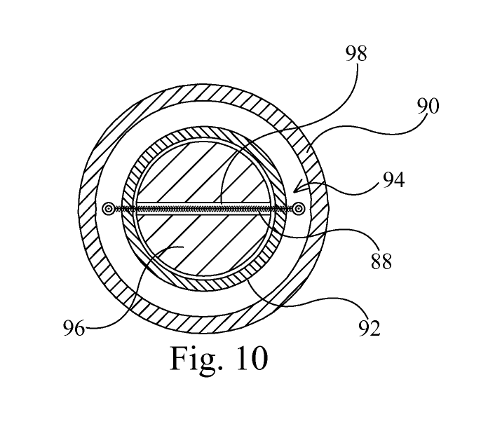

Referring now to FIGS. 6 to 10, there is shown a second embodiment of a medical instrument 80 for use during catheterization. The second embodiment of the medical instrument 80 comprises a syringe 82, a needle 84, a dilator 86, and a guidewire 88. The second embodiment of the medical instrument 80 is accordingly generally similar to the first embodiment of the medical instrument 10 with the following notable exceptions. The syringe 82 includes an outer barrel 90 and an inner barrel 92 with an annulus 94 between the outer barrel 90 and the inner barrel 92. A plunger 96 of the syringe 82 is reciprocatingly received by the inner barrel 92 as is conventional. The outer barrel 90 is longer than the inner barrel 92 and the guidewire 88 is able to extend along the annulus 94 in a U-shape configuration by passing around an end of the inner barrel 92 and through a through hole 98 in the plunger 96 of the syringe 82.

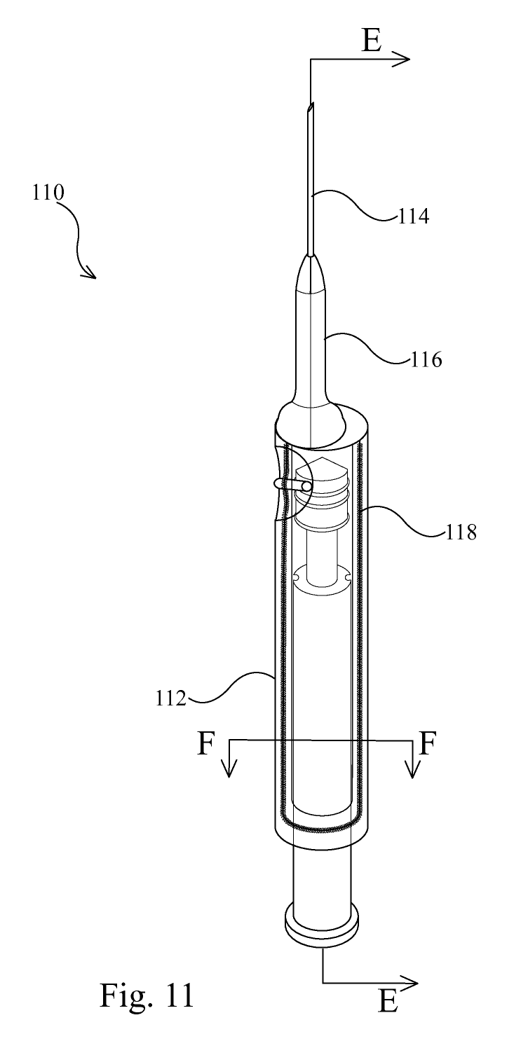

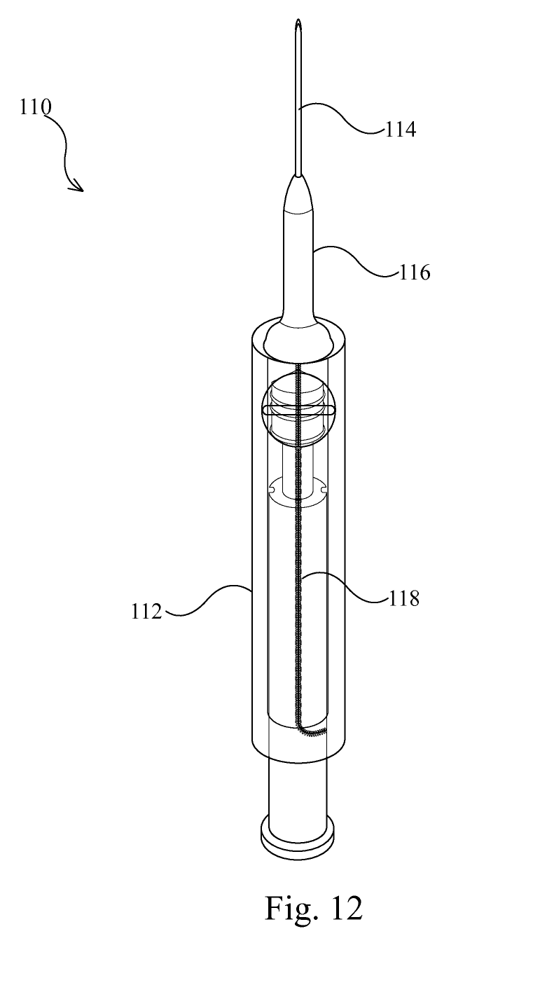

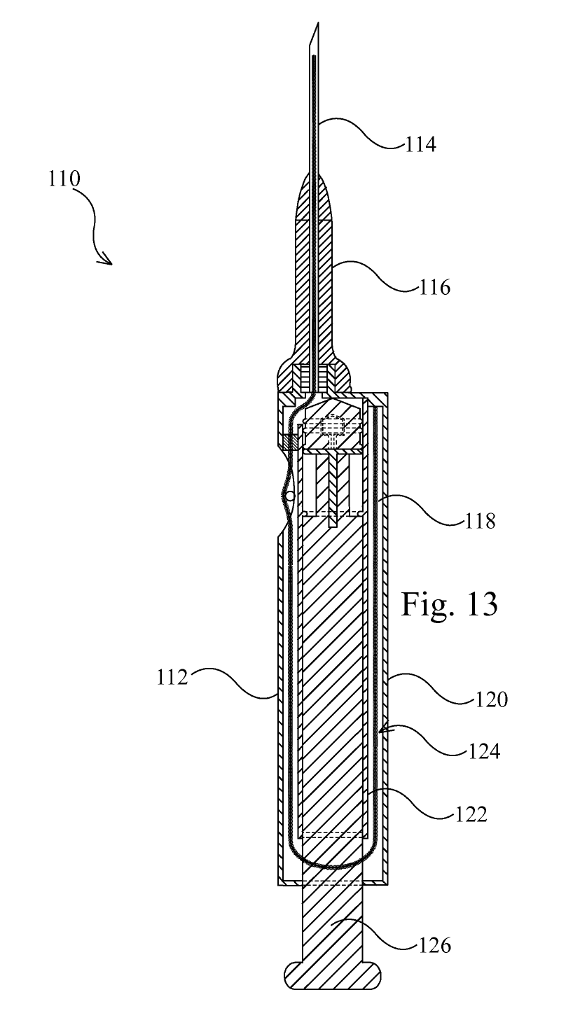

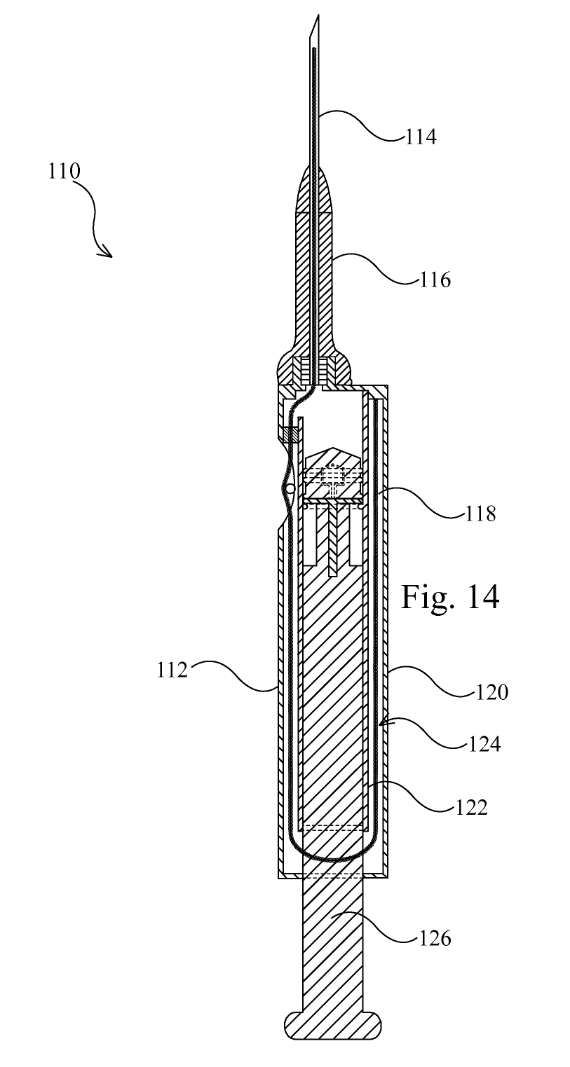

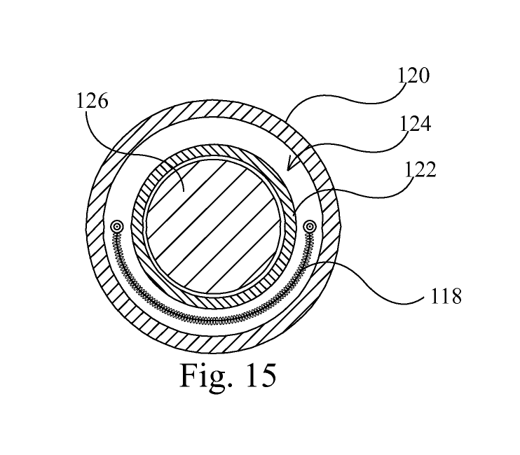

Referring now to FIGS. 11 to 15, there is shown a third embodiment of a medical instrument 110 for use during catheterization. The third embodiment of the medical instrument 110 comprises a syringe 112, a needle 114, a dilator 116, and a guidewire 118. The syringe 112 includes an outer barrel 120 and an inner barrel 122 with an annular space 124 between the outer barrel 120 and the inner barrel 122. A plunger 126 of the syringe 112 is reciprocatingly received by the inner barrel 122 as is conventional. The outer barrel 120 is longer than the inner barrel 122 and the guidewire 118 is able to extend along the annular space 124 in a U-shape configuration. The third embodiment of the medical instrument 110 is accordingly generally similar to the second embodiment of the medical instrument 80 with the following notable exception. There is no through hole in the plunger 126. Rather the guidewire 118 passes around both an end of the inner barrel 122 and the plunger 126.

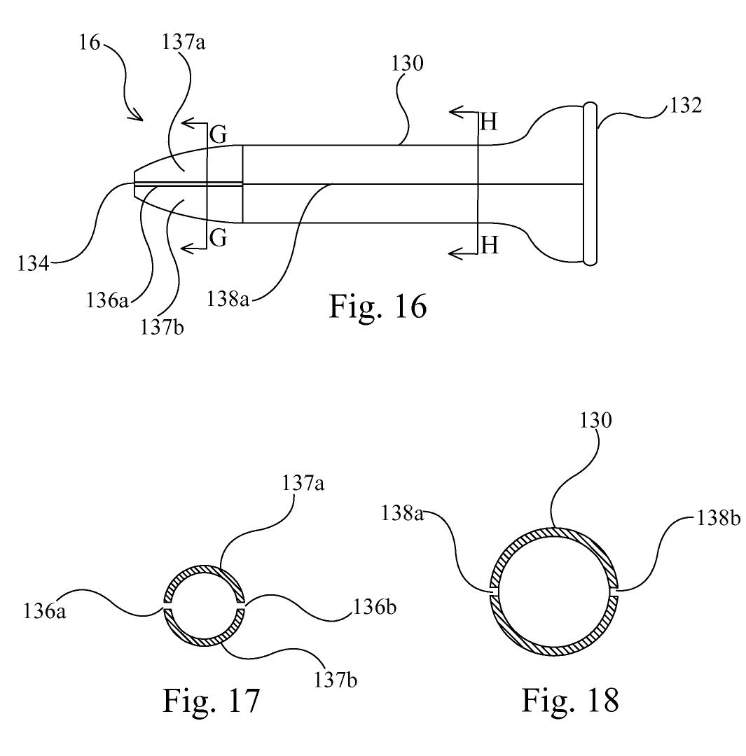

The dilator 16 of the first embodiment of the medical instrument 10 is shown in FIGS. 16 to 18. It will be understood by a person skilled in the art that the dilators of the second and third embodiments of the medical instrument are identical and accordingly are not described in detail herein. The dilator 16 is made of medical grade material such as polyvinyl chloride, PEBAX®, or another suitable material. The dilator 16 has an elongate body 130 with a connector 132 and a tip 134 at opposite ends thereof. The connector 132 is adapted to fit over the nozzle 74 of the syringe 12 as shown in FIGS. 3 and 4. Referring back to FIGS. 16 to 18, the dilator 16 gradually tapers from the connector 132 to the tip 134 to allow for progressive dilation during catheterization. An inner diameter of the dilator 16 near the connector 132 thereof is also greater than an inner diameter of the dilator 16 near the tip 134 thereof. The dilator 16 is also provided with longitudinal gaps 136a and 136b near the tip 134 thereof. The longitudinal gaps 136a and 136b separate tip portions 137a and 137b of the dilator 16 and render the dilator 16 dilatable at the tip portions 137a and 137b to facilitate manipulation of the dilator 16 during catheterization. The dilator 16 is further provided with longitudinal lines of weakness 138a and 138b which allow the dilator to be easily removed during catheterization. The dilator 16 may be peeled apart along the longitudinal lines of weakness 138a and 138b, allowing the dilator 16 to be removed in place rather than requiring the dilator to slide along the guidewire during catheterization. The longitudinal gaps 136a and 136b and the longitudinal lines of weakness 138a and 138b are axially aligned.

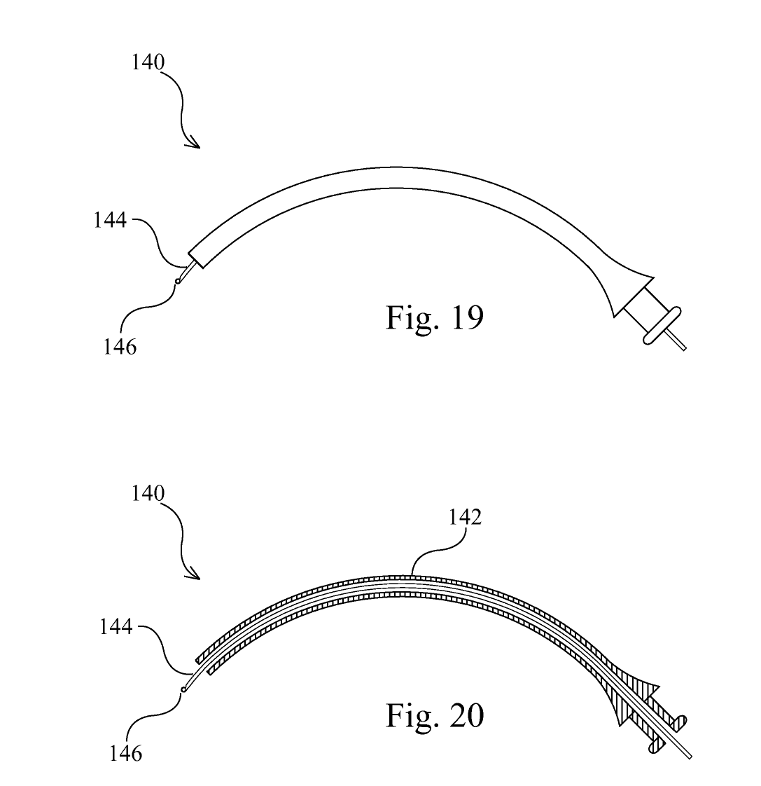

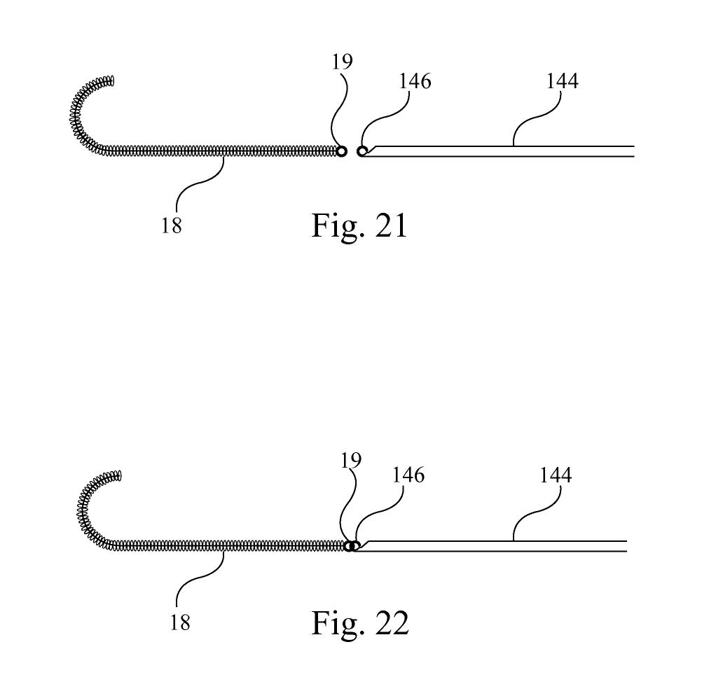

Referring now to FIGS. 19 and 20, there is shown a first embodiment of an improved catheter 140 for use with the first embodiment of the medical instrument 10. It will be understood however by a person skilled in the art that the catheter 140 may also be used with the second and third embodiments of the medical instrument in a similar manner. The catheter 140 has a lumen 142 and, as thus far described, is generally conventional. However, there is a guidewire 144 disposed within the lumen 142 of the catheter 140. The guidewire 144 extends axially beyond the lumen 142 of the catheter 140 and is provided with a connector which, in this example, is a hook 146 at an end thereof. Referring now to FIGS. 21 and 22, the hook 146 of the guidewire 144 of the catheter 140 is configured to engage with a connector which, in this example, is a loop 19 at an end of the guidewire 18 of the first embodiment of the medical instrument 10, and thereby couple the guidewires of the catheter and medical instrument. In this example, the connectors are hook and loop connectors but any suitable type of connectors may be used.

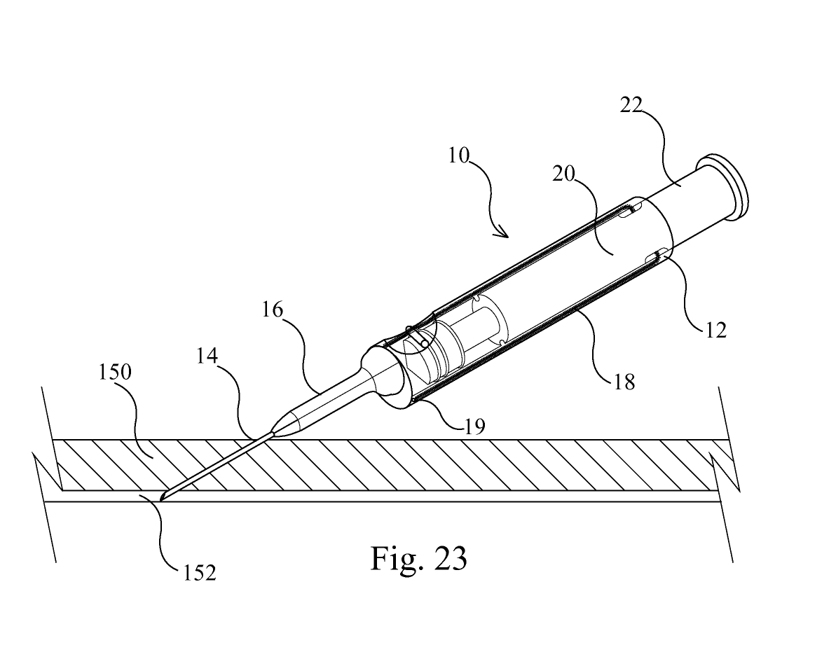

Use of the first embodiment of the medical instrument 10 and the catheter 140 during catheterization is shown in FIGS. 23 to 32.

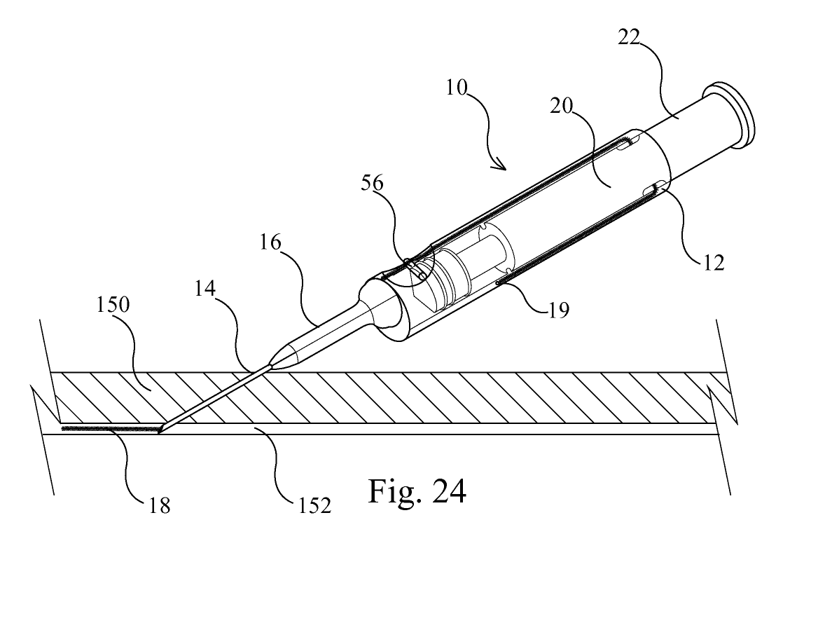

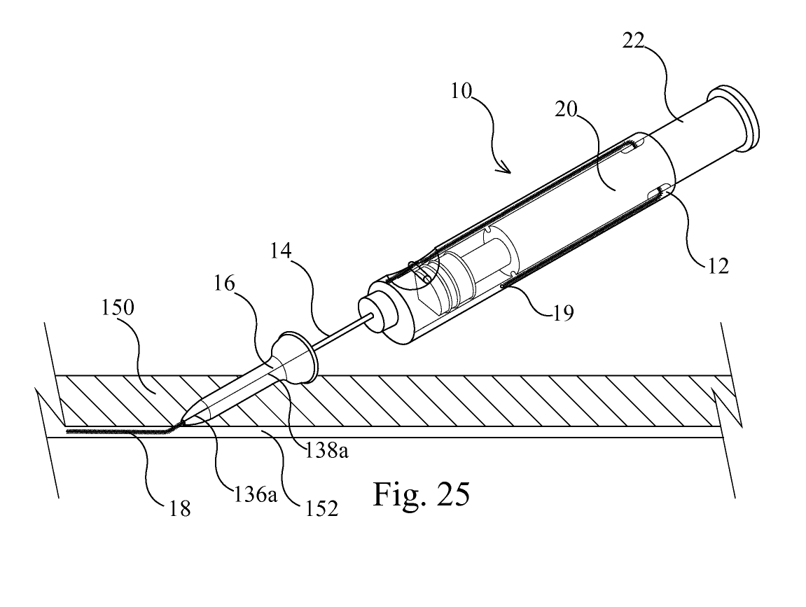

FIG. 23 shows the needle 14 of the medical instrument 10 inserted into patient tissue 150 and puncturing a blood vessel 152 which, in this example, is the subclavian vein. Blood may be aspirated into the barrel 20 of the syringe 12, by retracting the plunger 22 outwardly of the barrel, in order to confirm that the blood vessel 152 has in fact been punctured. The guidewire 18 of the medical instrument 10 is inserted into the blood vessel 152, as shown in FIG. 24, by advancing the guidewire 18 through the needle 14. This is done manually by advancing the guidewire 18 over the roller 56 to project the guidewire 18 out of the syringe 12, through the needle 14, and into the blood vessel 152. The dilator 16 is then inserted into the tissue 150 around the guidewire 18 while the needle 14 and syringe 12 are simultaneously withdrawn as shown in FIG. 25. The guidewire 18 is retained in the dilator 16 by pinching the dilator 16 to hold the guidewire 18 in place as the needle 14 and syringe 12 are withdrawn leaving only the guidewire 18 and dilator 16 in the tissue 150.

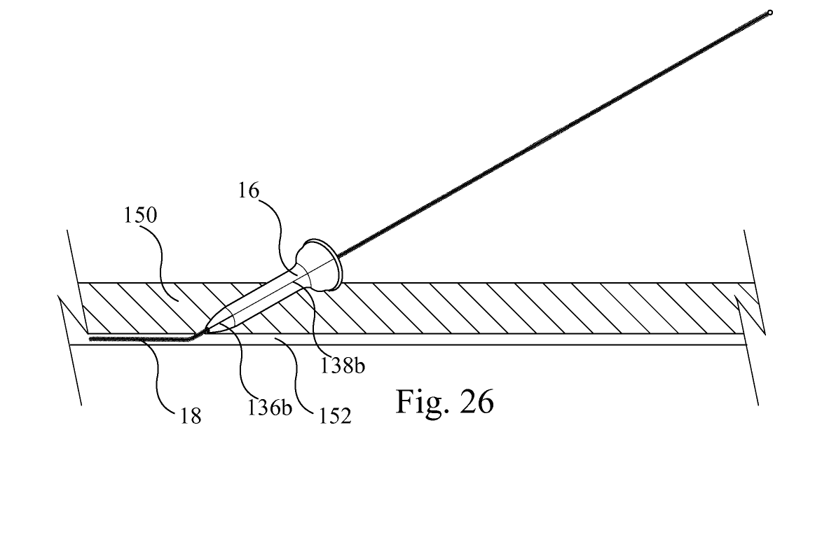

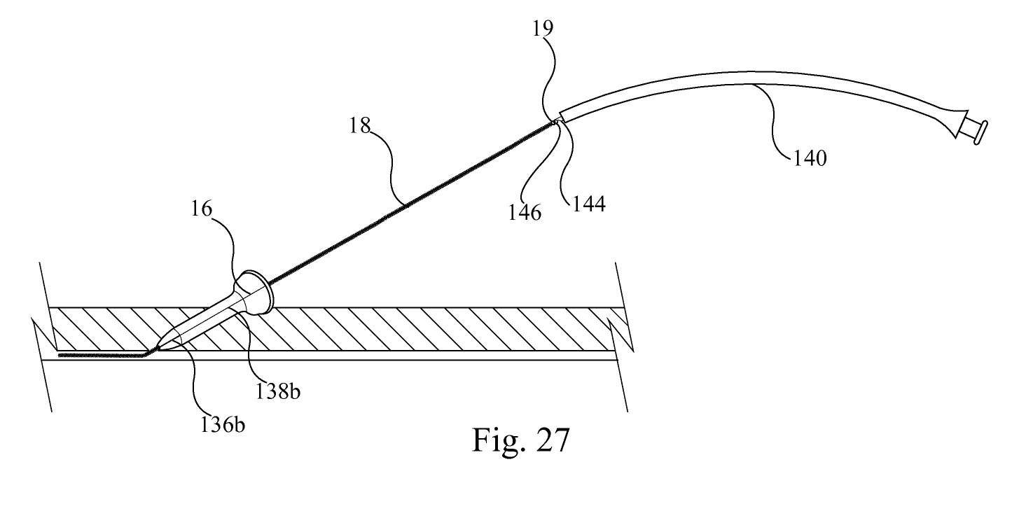

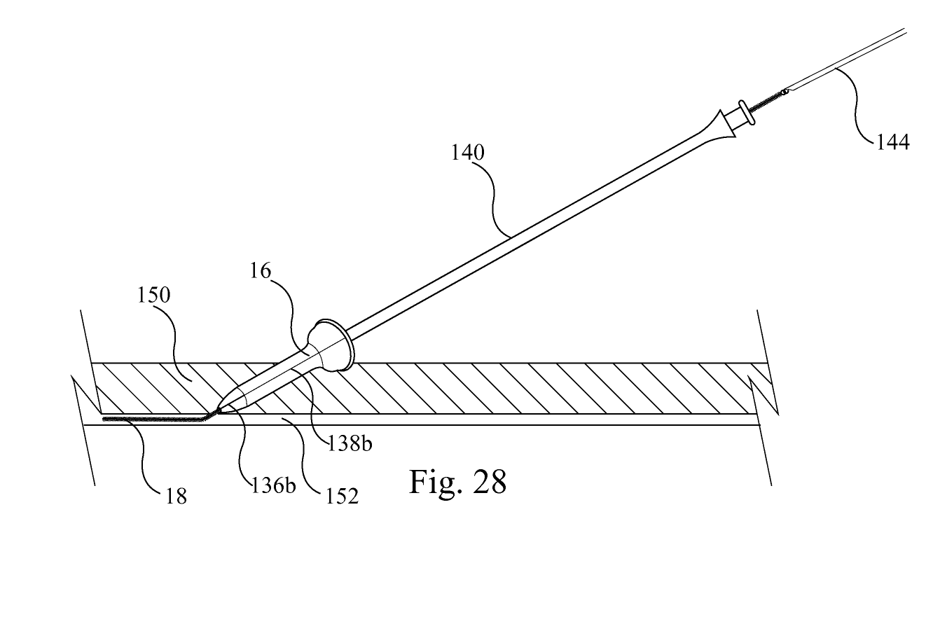

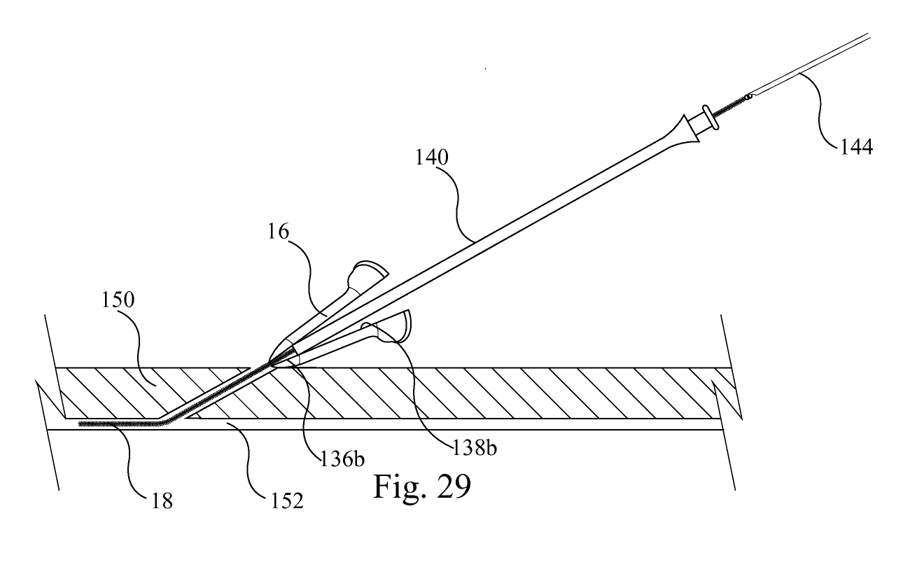

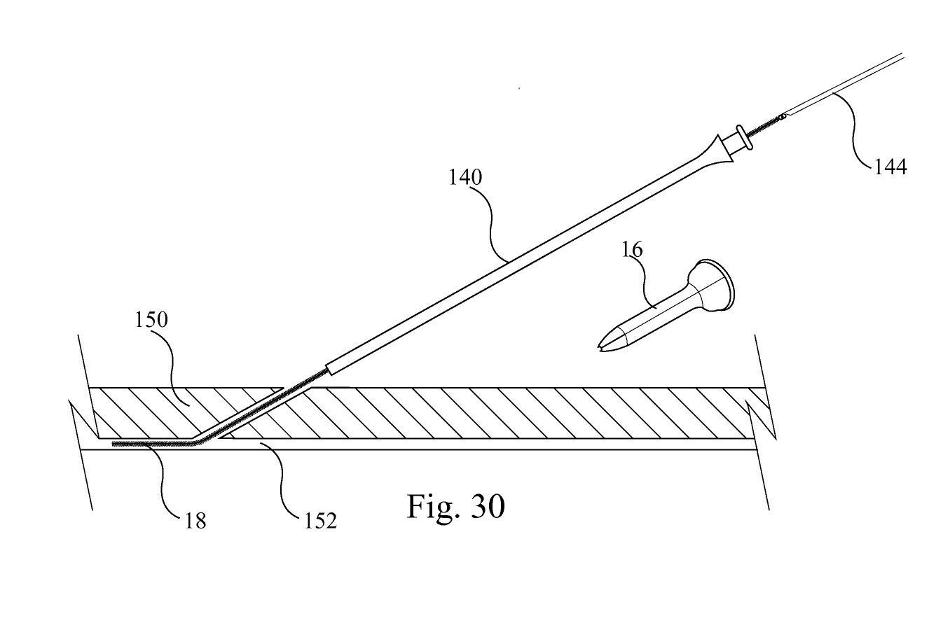

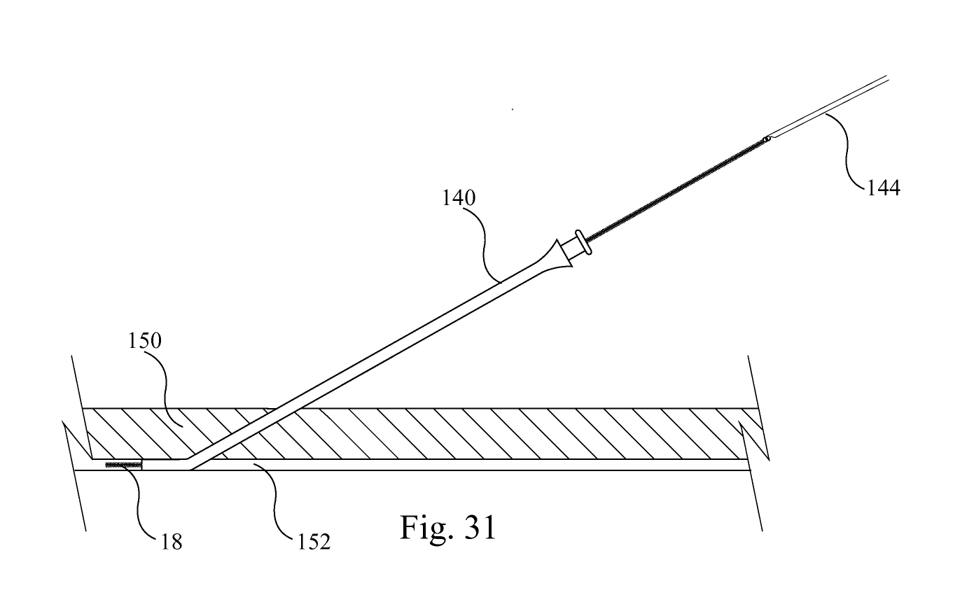

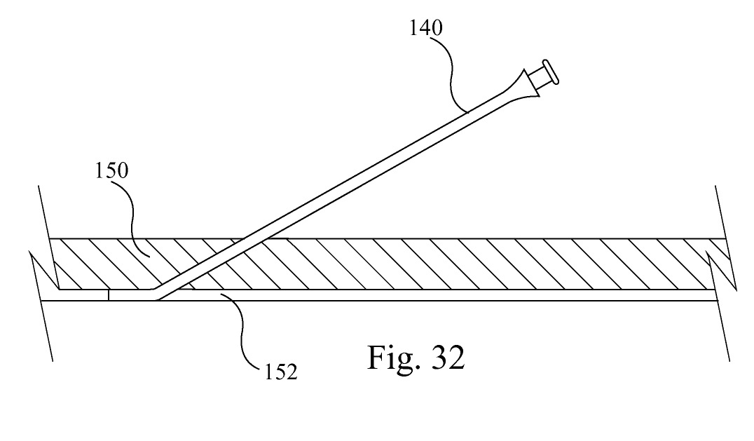

FIGS. 26 and 27 respectively show the dilator 16 being rotated to dilate the tissue 150 surrounding the guidewire 18 and the guidewire 18 being coupled to the guidewire 144 of the catheter 140. In this example, the dilator 16 has been rotated substantially one hundred and eighty-degrees from the position shown in FIG. 25. These two steps may be done in any order and, once both of the aforementioned steps are completed, the catheter 140 is moved along the guidewires until the catheter 140 is received by the dilator 16 as shown in FIG. 28. The dilator 16 is then removed, as shown in FIGS. 29 and 30, by peeling the dilator 16 off the guidewire 18 along the lines of weakness 138a and 138b. The catheter 140 is then inserted in the blood vessel 152 as shown in FIG. 31. Finally, the guidewire 18 is withdrawn from the catheter 140 and catheterization is complete as shown in FIG. 32.

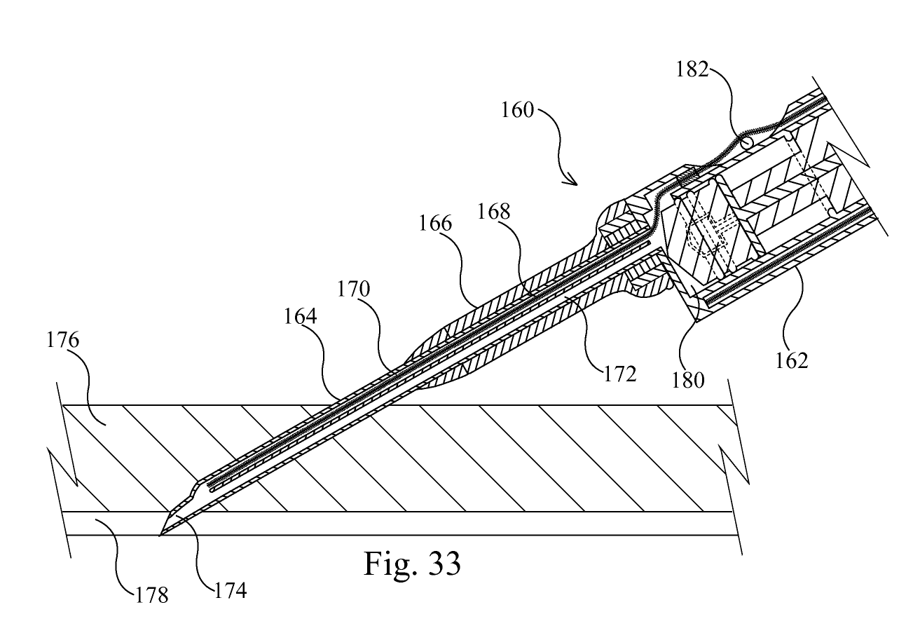

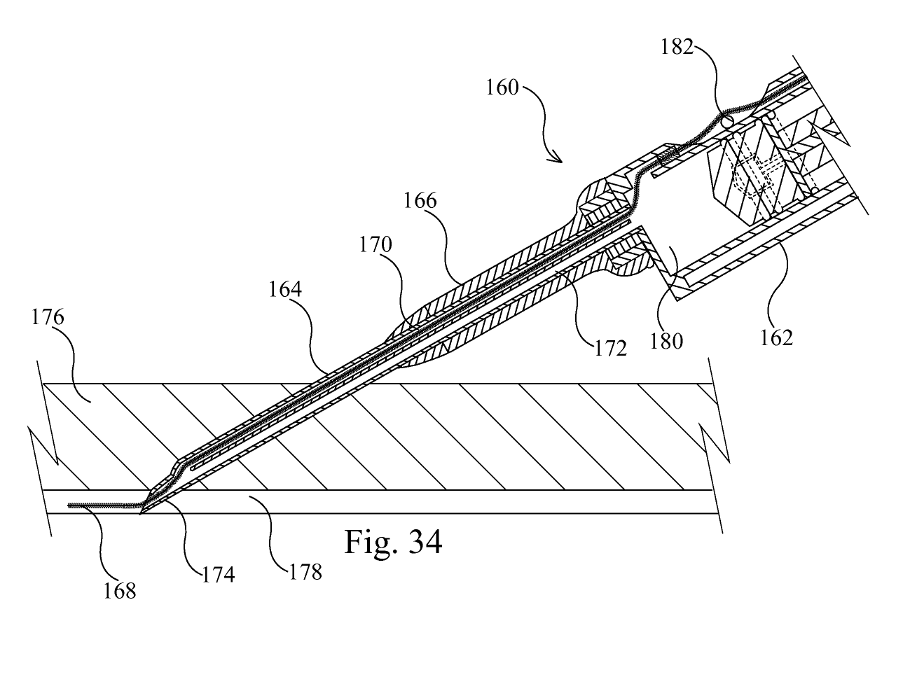

Referring now to FIGS. 33 and 34, there is shown a fourth embodiment of a medical instrument 160 for use during catheterization. The fourth embodiment of the medical instrument 160 comprises a syringe 162, a needle 164, a dilator 166, and a guidewire 168. The fourth embodiment of the medical instrument 160 is accordingly generally similar to the first embodiment of the medical instrument 10 with the following notable exception. The needle 164 includes a first lumen 170 and a second lumen 172 which are substantially parallel to each other in this example. The first lumen 170 and the second lumen 172 converge at a tip 174 of the needle 164. The guidewire 168 extends into the first lumen 170 in this example. The needle 164 is inserted into patient tissue 176 and punctures a blood vessel 178 which, in this example, is the subclavian vein. Blood may be aspirated into a barrel 180 of the syringe 162 via the second lumen 172 of the needle 164, by retracting a plunger (not shown) outwardly of the barrel, in order to confirm that the blood vessel 178 has in fact been punctured. The guidewire 168 is inserted into the blood vessel 178, as shown in FIG. 34, by advancing the guidewire 168 through the first lumen 170 of the needle 164. This is done manually by advancing the guidewire 168 over a roller 182 to project the guidewire 168 out of the syringe 162, through the first lumen 170 of the needle 164, and into the blood vessel 178. The catheter 140 is then introduced along the guidewire 168 and into the blood vessel 178 in a similar manner as described above for the first embodiment of the medical instrument 10.

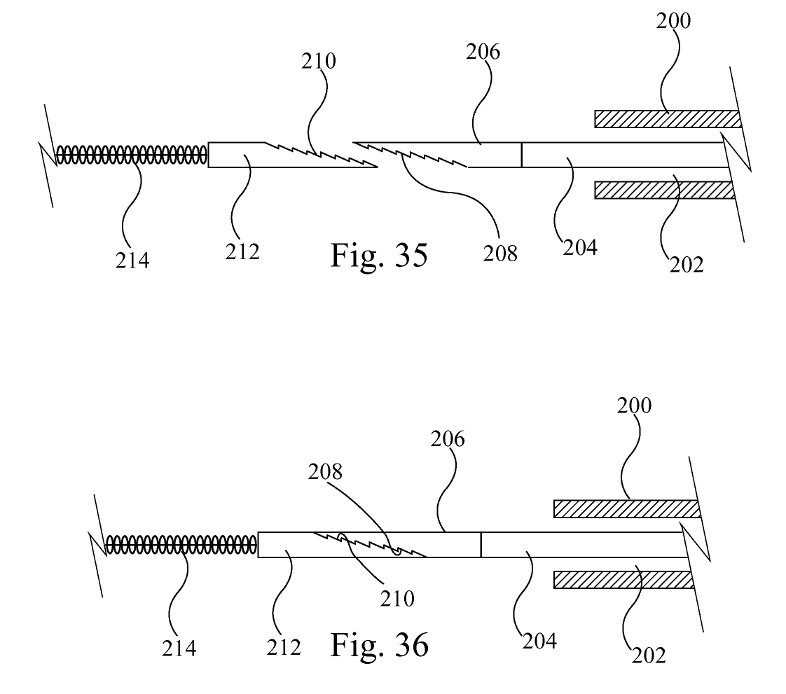

FIGS. 35 and 36 show a second embodiment of an improved catheter 200. The catheter 200 has a lumen 202 and a guidewire 204 disposed within and extending axially beyond the lumen. The catheter 200 is accordingly generally similar to the catheter 140 with the following notable exception. The guidewire 204 is provided with a connector 206 at an end thereof which has a serrated edge 208. The serrated edge 208 is shaped to engage with a serrated edge 210 of a connector 212 at an end of another embodiment of a guidewire 214 of the first embodiment of the medical instrument 10, and thereby couple the guidewires of the catheter and medical instrument. It will be understood however by a person skilled in the art that the guidewire 214 may also be used with the second, third and fourth embodiments of the medical instrument. The connectors 206 and 212 may be formed of a magnetic material to allow their respective serrated edges 208 and 210 to be magnetically connected to each other.

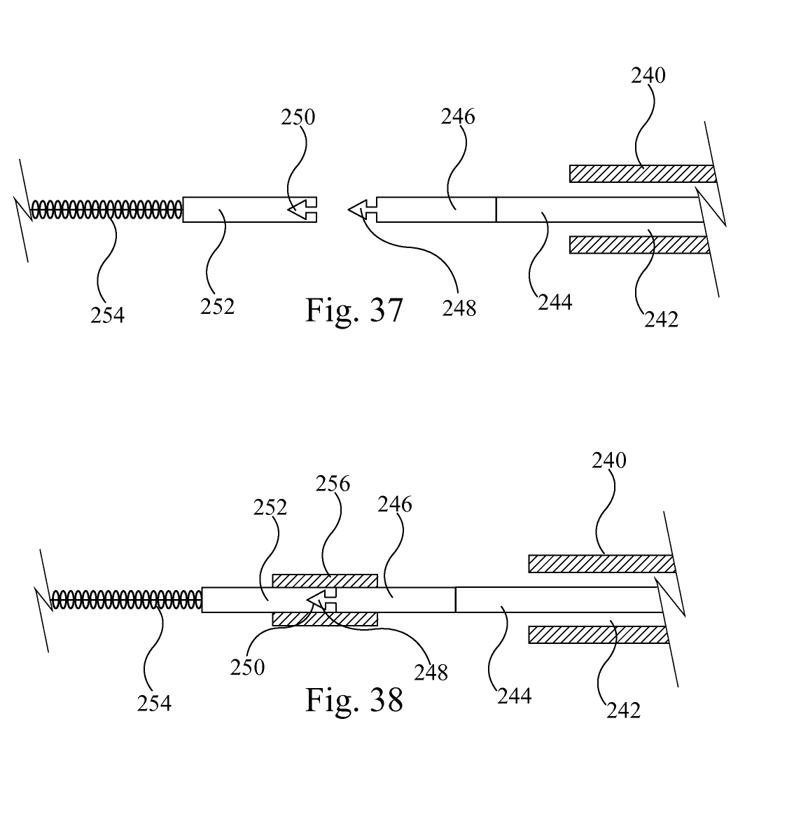

Referring now to FIGS. 37 and 38, there is shown a third embodiment of an improved catheter 240. The catheter 240 has a lumen 242 and a guidewire 244 disposed within and extending axially beyond the lumen. The catheter 240 is accordingly generally similar to the catheter 140 with the following notable exception. The guidewire 244 is provided with a connector 246 at an end thereof which has a projection 248. The projection 248 in this example is generally in the shape of an arrow but may be other shapes. The projection 248 engages an opening 250, which is complementary in shape to the projection 248, in a connector 252 at an end of another embodiment of a guidewire 254 of the first embodiment of the medical instrument 10, and thereby couples the guidewires of the catheter and medical instrument. It will be understood however by a person skilled in the art that the guidewire 244 may also be used with the second, third and fourth embodiments of the medical instrument. The connectors 246 and 252 may be formed of a magnetic material to allow the projection 248 and the opening 250 to be magnetically connected to each other. There may be a sleeve 256 covering the connection between the projection 248 of the connector 246 and the opening 250 in the connector 252.

It will be understood by a person skilled in the art that many of the details provided above are by way of example only, and are not intended to limit the scope of the invention which is to be determined with reference to the following claims.

What is claimed is:

1. A central venous catheter kit comprising:
(a) an instrument including:
a syringe having a barrel and a plunger;
a needle extending axially from the syringe, the needle having a lumen and the lumen of the needle being in fluid communication with the barrel of the syringe;
a dilator disposed about the needle;
a guidewire substantially disposed within the syringe and a guide for advancing the guidewire out of the syringe through the lumen of the needle;
and
(b) a catheter having a lumen and a guidewire disposed within the lumen of the catheter;
wherein the guidewire substantially disposed within the syringe and the guidewire disposed within the lumen of the catheter are each provided with a connector to allow said guidewires to be coupled.

2. The central venous catheter kit as claimed in claim 1 wherein the instrument further includes a channel in the barrel of the syringe and wherein the guidewire substantially disposed within the syringe extends along the channel.

3. The central venous catheter kit as claimed in claim 1 wherein the instrument further includes a first channel and a second channel in the barrel of the syringe and wherein the guidewire substantially disposed within the syringe extends along the first channel and the second channel.

4. The central venous catheter kit as claimed in claim 3 wherein the instrument further includes a through hole in the plunger of the syringe and the guidewire substantially disposed within the syringe extends along the first channel, through the through hole and along the second channel.

5. The central venous catheter kit as claimed in claim 4 wherein the instrument further includes a detent to limit how far the plunger can be withdrawn from the barrel so the plunger does not interfere with the guidewire.

6. The central venous catheter kit as claimed in claim 1 wherein the barrel of the syringe of the instrument includes an inner barrel and an outer barrel and wherein the guidewire substantially disposed within the syringe extends between the inner barrel and the outer barrel.

7. The central venous catheter kit as claimed in claim 6 wherein the instrument further includes a through hole in the plunger of the syringe and the guidewire substantially disposed within the syringe extends in a U-shaped configuration within the syringe and through the through hole.

8. The central venous catheter kit as claimed in claim 7 wherein the instrument further includes a detent to limit how far the plunger can be withdrawn from the barrel so the plunger does not interfere with the guidewire.

9. The central venous catheter kit as claimed in claim 6 wherein the guidewire substantially disposed within the syringe extends in a U-shaped configuration within the syringe and around the plunger.

10. The central venous catheter kit as claimed in claim 1 wherein the dilator of the instrument includes longitudinal lines of weakness and longitudinal gaps which are aligned with the longitudinal lines of weakness.

11. The central venous catheter kit as claimed in claim 1 wherein the needle of the instrument includes a first lumen and a second lumen which is substantially parallel to the first lumen and wherein the guidewire substantially disposed within the syringe is advanced out of the syringe through the first lumen of the needle.

12. The central venous catheter kit as claimed in claim 1 wherein the guidewire disposed within the lumen of the catheter extends axially beyond the lumen.

13. The central venous catheter kit as claimed in claim 1 wherein the connectors of the guidewire substantially disposed within the syringe and the guidewire disposed within the lumen of the catheter are formed of a magnetic material.

14. An instrument comprising:
a syringe having a barrel and a plunger;
a needle extending axially from the syringe, the needle having a lumen and the lumen of the needle being in fluid communication with the barrel of the syringe;
a dilator disposed about the needle, the dilator having longitudinal lines of weakness and longitudinal gaps which are aligned with the longitudinal lines of weakness; and
a guidewire substantially disposed within the syringe and a guide for advancing the guidewire out of the syringe through the lumen of the needle.

15. The instrument as claimed in claim 14 further including a channel in the barrel of the syringe and wherein the guidewire extends along the channel.

16. The instrument as claimed in claim 14 further including a detent to limit how far the plunger can be withdrawn from the barrel so the plunger does not interfere with the guidewire.

17. The instrument as claimed in claim 14 further including:
a first channel in the barrel of the syringe;
a second channel in the barrel of the syringe; and
a through hole in the plunger of the syringe and wherein the guidewire extends along the first channel, through the through hole and along the second channel.

18. The instrument as claimed in claim 14 wherein the barrel of the syringe includes an inner barrel and an outer barrel and wherein the guidewire extends between the inner barrel and the outer barrel.

19. The instrument as claimed in claim 18 wherein the guidewire extends in a U-shaped configuration within the syringe and around the plunger.

20. The instrument as claimed in claim 18 further including a through hole in the plunger of the syringe and wherein the guidewire extends in a U-shaped configuration within the syringe and through the through hole.

21. The instrument as claimed in claim 14 wherein the needle includes a first lumen and a second lumen which is substantially parallel to the first lumen and wherein the guidewire substantially disposed within the syringe is advanced out of the syringe through the first lumen of the needle.

22. A catheter having a lumen and a guidewire disposed within the lumen of the catheter, wherein the guidewire extends axially beyond the lumen.
